(19) United States
(12) Patent Application Publication
(10) Pub. No.: US 2018/0326023 A1
(43) Pub. Date: Nov. 15, 2018

(54) PLASMINOGEN DOSAGE REGIMEN FOR WOUND HEALING

(71) Applicant: Prometic Biotherapeutics, Ltd., Cambridge (GB)

(72) Inventors: ULF BERTHEIM, Umeå (SE); TOR NY, Umeå (SE); PIERRE LAURIN, Ville Mont-Royal (CA); MARTIN ROBITAILLE, Saint-Colomban (CA)

(21) Appl. No.: 15/774,155

(22) PCT Filed: Nov. 10, 2016
(86) PCT No.: PCT/IB2016/001606

§ 371 Date: May 7, 2018

Related U.S. Application Data
(60) Provisional application No. 62/253,352, filed on Nov. 10, 2015.

(51) Int. Cl. A61K 38/48; A61P 17/02

(52) U.S. Cl. CPC A61K 38/484; A61K 9/0019; A61P 17/02

(57) ABSTRACT

The invention concerns the use of plasminogen or a biologically active variant thereof for the preparation of a medicament for local administration of at least one dose of plasminogen or a biologically active variant thereof for promoting the healing of a wound in a subject. The dose is between about 2 mg and about 30 mg. The frequency of the administration may vary from once a day to once a week. The invention further concerns the related medicaments and methods of treatment.

### FIELD OF INVENTION

The present invention relates to the field of medicine, and more particularly to wound healing, including for example treatment of an acute or a chronic wound.

### BACKGROUND OF INVENTION

Wound healing or wound repair is a complex mechanism that involves three stages: inflammation, new tissue formation, and remodelling (Gurtner et al. 2008 Nature. Vol. 453, No. 15, pp. 314-321). In some cases, the mechanism of repair is impaired and the wound never heals. For example, this occurs frequently in diabetic patients, who often suffer from nephropathy, impaired vasoregulation and a weakened immune system that make it harder for wounds to heal. For diabetic patients, small injuries may progress to larger wounds because of reduced healing capacity. A wound can become chronic when it does not heal in a predictable amount of time. Wounds that do not heal within three months are typically considered chronic. A common wound for diabetic patients is the diabetic foot ulcer, which is very disabling and frequently leads to amputation of the leg, and a higher risk of mortality (Jeffcoate and Harding. The Lancet. Review: Diabetic Foot Ulcers, published online Feb. 25, 2003). The only known treatments for diabetic foot ulcer include infection management, off-loading techniques, different types of bandage and amputation.

Another type of chronic wound is observed in hospitalized persons that are bedridden for a long period of time, and are usually called bed sores or pressure sores or pressure ulcers, which are characterized by four stages. At stage 1, the sore is not an open wound but the skin is red and warm and may be painful. At stage 2, the skin breaks open, wears away, or forms an ulcer, which is usually tender and painful. At stage 3, the sore gets worse and extends into tissue beneath the skin, forming a small crater. At stage 4, the pressure sore is very deep, reaching into muscle and bone and causing extensive damage. At stages 3 and 4, there may be little or no pain due to significant tissue damage. Commonly used treatments for pressure sores over the years have included innovative mattresses, ointments, creams, solutions, dressings, ultrasonography, ultraviolet heat lamps, sugar, and surgery. In choosing a treatment strategy, consideration should be given to the stage of the wound and the purpose of the treatment (e.g., protection, moisture, or removal of necrotic tissue). For stage 3 and 4 lesions, surgical intervention (e.g., flap reconstruction) may be required, though some of these lesions must be treated conservatively (without surgery) because of coexisting medical problems and the patient cannot undergo this procedure.

Another type of chronic wound is a vascular wound, which could be venous or arterial. Typical vascular wound is venous leg ulcer and venous leg ulcer. A leg ulcer is frequently a chronic wound that lasts for more than four to six weeks to heal. Leg ulcers usually develop on the inside of the leg, just above the ankle. The symptoms of a venous leg ulcer include pain, itching and swelling in the affected leg. There may also be discolored or hardened skin around the ulcer, and the sore may produce a foul-smelling discharge. Just like the diabetic foot ulcer, leg ulcer can present themselves either/or in combination with arterial leg ulcers; caused by poor blood circulation in the arteries, diabetic leg ulcers; caused by the high blood sugar associated with diabetes, vasculitis leg ulcers; associated with chronic inflammatory disorders such as rheumatoid arthritis and lupus, traumatic leg ulcers; caused by injury to the leg and malignant leg ulcers; caused by a tumour of the skin of the leg.

Acute and/or chronic wounds are typically categorized as either arterial (ischemic) ulcers like diabetic foot ulcers; venous wounds (varicose, leg, stasis) ulcers; lymphedema (chronic dermal disruption) ulcers; pressure (decubitus) ulcers; due to late effect of trauma; an uncontrolled infection and/or an autoimmune ulcer such as but limited to psoriasis and lupus. Typically these wounds or ulcers have vascular compromise (arterial, venous, microvascular). In addition, they have reduced tissue oxygen concentration and they have metabolic compromise.

Single etiology ulcers are uncommon and wounds or ulcers intended to be treated by this application could have mixed etiology such as diabetic/pressure ulcer, pressure/venous ulcer, venous/arterial ulcer or pressure/venous/arterial ulcer.

Another type of chronic wound is observed in tympanic membrane perforation. In some patients, a tympanic membrane perforation heals normally; but for others, the perforation never closes. Infection is the principal result (or side effect) of tympanic membrane perforation (TMP). Acute infection of the middle ear may cause a relative ischemia in the drum concurrent with increased pressure in the middle ear space. This leads to a tear or rupture of the eardrum that is usually preceded by severe pain. If the perforation does not heal, it leaves a residual tympanic membrane perforation. Ear canal infections rarely cause tympanic membrane perforations. When this occurs, it is often associated with infection by Aspergillus niger. Traumatic perforations may also result from blows to the ear (eg, being struck with the flat of the hand; falling from water skis with the head hitting the water surface, ear down). Exposure to severe atmospheric overpressure from an explosion can tear the drum. Tympanic membrane perforation from water pressure occurs in scuba divers, usually in a drum with atrophy from previous disease. Objects used to clean the ear canal can perforate the drum. Evidence exists that such perforations are less likely to heal spontaneously. Tympanic membrane perforation is intentionally created whenever a surgeon makes an incision in the eardrum (myringotomy). When pressure-equalizing tubes (ventilating tubes) are placed, the tympanic membrane perforation purposely is held open. Failure of surgically created openings to heal when the tube extrudes results in chronic tympanic membrane perforation. Perforation symptoms may include audible whistling sounds during sneezing and nose blowing, decreased hearing, and a tendency to infection during colds and when water enters the ear canal. Many persons live their lives with tympanic membrane perforations that are entirely without symptoms. However, perforations may be associated with recurrent infection when exposed to water.

Ligneous conjunctivitis is a rare form of chronic conjunctivitis characterized by recurrent, fibrin-rich pseudomembranous lesions (wound) of wood-like consistency that develop mainly on the underside of the eyelid (tarsal conjunctiva). It is generally a systemic disease which may involve the periodontal tissue, the upper and lower respiratory tract, kidneys, middle ear, and female genitalia. It can be sight-threatening, and death can occasionally occur from pulmonary involvement. This medical condition can present itself as either/both an acute or chronic wound.

There is currently no effective treatment for chronic wounds such as diabetic foot ulcers or deep pressure sores, other than surgery (amputation or reconstruction); and there is no treatment for chronic wounds such tympanic membrane perforations.

There is thus a need for novel methods for improving or promoting wound healing, including for the non-invasive treatment of acute and chronic wounds.

The present description refers to a number of documents, the content of which is herein incorporated by reference in their entirety.

### BRIEF SUMMARY OF THE INVENTION

The present invention relates to the uses of plasminogen or a biologically active variant thereof, for promoting wound healing.

The present invention relates to the following claims 1 to 66:

1. Use of plasminogen or a biologically active variant thereof for the preparation of a medicament for promoting the healing of a wound in a subject, wherein the medicament is adapted for local administration and comprises at least one dose of plasminogen or a biologically active variant thereof, of about 2 mg to about 30 mg.

2. The use of item 1, wherein the local administration is intracutaneous, subcutaneous, intramuscular or topical administration.

3. The use of item 2, wherein the local administration is performed around the wound, in vicinity of the wound or in the wound.

4. The use of item 2, wherein the local administration consists of several injections performed around the wound at about 0.5 cm thereof.

5. The use of item 1, wherein the wound is an opened wound.

6. The use of item 1, wherein the wound is a chronic wound or an acute wound.

7. The use of any one of items 1 to 6, wherein the dose contains about 2 mg, about 4 mg, about 5 mg, about 10 mg, about 15 mg, about 20 mg, about 25 mg, or about 30 mg of plasminogen or a biologically active variant thereof.

8. The use of item 1, wherein the local administration is performed at a frequency of at least once a week, at least twice-a-week, at least every-three-day, at least every-two-day, at least once a day, or at least twice-a-day.

9. The use of item 1 or 8, wherein the medicament comprises a plurality of doses of plasminogen or a biologically active variant thereof, for performing multiple administrations until the wound size is reduced by at least 50%.

10. The use of item 1 or 8, wherein the medicament comprises a plurality of doses of plasminogen or a biologically active variant thereof, for performing multiple administrations until the wound size is reduced by at least 90%.

11. The use of item 1, wherein the plasminogen or biologically active variant thereof is in a solution at a concentration from about 1 mg/ml to about 20 mg/ml.

12. The use of item 11, wherein the concentration is of about 1 mg/ml, 5 mg/ml, about 10 mg/ml, about 15 mg/ml or about 20 mg/ml.

13. The use of item 1, wherein the wound has an area, and wherein the dose of plasminogen or a biologically active variant thereof is proportional with said area.

14. The use of item 13, wherein the dose is expressed in mg and corresponds to about 2.5 times the wound area expressed in cm².

15. The use of item 1, wherein the wound has an area, and wherein the dose is about 10 mg when said wound area is less than 4 cm²; the dose is about 20 mg when said wound area is from 4 cm² to 8 cm², or the dose is about 30 mg when said wound area is greater than 8 cm².

16. The use of item 1, wherein the wound has a circumference, and wherein the dose of plasminogen or a biologically active variant thereof is proportional with said wound circumference.

17. The use of item 16, wherein the dose is expressed in mg and corresponds to about 1.5 times the wound circumference expressed in cm.

18. The use of item 4, wherein said several injections consist of 2 to 30 injections.

19. The use of item 1, wherein the subject is a mammal, and preferably a human.

20. The use of item 1, wherein the wound is a chronic wound selected from the group of a diabetic foot ulcer, a diabetic wound, a pressure sore, a pressure ulcer, a tympanic membrane perforation, a burn wound, an incision wound, a venous leg ulcer, an arterial leg ulcer, a vascular wound or a lesion.

21. A medicament for promoting the healing of a wound in a subject, said medicament is adapted for local administration and comprises at least one dose of plasminogen or a biologically active variant thereof, of about 2 mg to about 30 mg.

22. The medicament of item 21, wherein the medicament further comprises a device for intracutaneous, subcutaneous, intramuscular or topical administration.

23. The medicament of item 21, wherein said dose is about 2 mg, about 4 mg, about 5 mg, about 10 mg, about 15 mg, about 20 mg, about 25 mg or about 30 mg of plasminogen or a biologically active variant thereof.

24. The medicament of item 21, wherein said plasminogen or a biologically active variant thereof is in the form of a powder ready for reconstitution.

25. The medicament of item 21, wherein the plasminogen or biologically active variant thereof is in a solution at a concentration of about 1 mg/ml to about 20 mg/ml.

26. The medicament of item 25, wherein the plasminogen or biologically active variant thereof is in a solution at a concentration of about 1 mg/ml, 5 mg/ml, about 10 mg/ml, about 15 mg/ml or about 20 mg/ml.

27. The medicament of item 22 wherein the device is adapted for performing several injections.

28. The medicament of item 27, wherein said several injections are performed around the wound, in vicinity of the wound or in the wound.

29. The medicament of item 21, wherein the local administration consists of several injections performed around the wound at about 0.5 cm thereof.

30. The medicament of item 21, wherein the wound is an opened wound.

31. The medicament of item 21, wherein the wound is a chronic wound or an acute wound.

32. The medicament of any one of items 21 to 31, wherein the dose contains about 2 mg, about 4 mg, about 5 mg, about 10 mg, about 15 mg, about 20 mg, about 25 mg, or about 30 mg of plasminogen or a biologically active variant thereof.

33. The medicament of item 21, wherein the local administration is performed at a frequency of at least once a week, at least twice-a-week, at least every-three-day, at least every-two-day, at least once a day, or at least twice-a-day.

34. The medicament of item 21 or 27, wherein the medicament comprises a plurality of doses of plasminogen or a biologically active variant thereof, for performing multiple administrations until the wound size is reduced by at least 50%.

35. The use of item 21 or 27, wherein the medicament comprises a plurality of doses of plasminogen or a biologically active variant thereof, for performing multiple administrations until the wound size is reduced by at least 90%.

36. The medicament of item 21, wherein the plasminogen or biologically active variant thereof is in a solution at a concentration from about 1 mg/ml to about 20 mg/ml.

37. The medicament of item 36 wherein the plasminogen or biologically active variant thereof is in solution at a concentration of about 1 mg/ml, 5 mg/ml, about 10 mg/ml, about 15 mg/ml or about 20 mg/ml.

38. The medicament of item 21, wherein the wound has an area, and wherein the dose of plasminogen or a biologically active variant thereof is proportional with said area.

39. The medicament of item 38, wherein the dose is expressed in mg and corresponds to about 2.5 times the wound area expressed in cm².

40. The medicament of item 21, wherein the wound has an area, and wherein the dose is about 10 mg when said wound area is less than 4 cm²; the dose is about 20 mg when said wound area is from 4 cm² to 8 cm², or the dose is about 30 mg when said wound area is greater than 8 cm².

41. The medicament of item 21, wherein the wound has a circumference, and wherein the dose of plasminogen or a biologically active variant thereof is proportional with said wound circumference.

42. The medicament of item 41, wherein the dose is expressed in mg and corresponds to about 1.5 times the wound circumference expressed in cm.

43. The medicament of item 27, wherein said several injections consist of 2 to 30 injections.

44. The medicament of item 21, wherein the subject is a mammal, and preferably a human.

45. The medicament of item 21, wherein the wound is a chronic wound selected from the group of a diabetic foot ulcer, a diabetic wound, a pressure sore, a pressure ulcer, a tympanic membrane perforation, a burn wound, an incision wound, a venous leg ulcer, an arterial leg ulcer, a vascular wound or a lesion.

46. The medicament of item 21, wherein said plasminogen or a biologically active variant thereof is in the form of a powder ready for reconstitution.

47. A method for promoting the healing of a wound in a subject, comprising the local administration of at least one dose of plasminogen or a biologically active variant thereof to said subject of about 2 mg to about 30 mg.

48. The method of item 41, wherein the local administration is intracutaneous, subcutaneous, intramuscular or topical administration.

49. The method of item 41, wherein the local administration consists of several injections.

50. The method of item 49, wherein said several injections are performed around the wound, in vicinity of the wound or in the wound.

51. The method of item 47, wherein the local administration consists of several injections performed around the wound at about 0.5 cm thereof.

52. The method of item 47, wherein the wound is an opened wound.

53. The method of item 47, wherein the wound is a chronic wound or an acute wound.

54. The method of any one of items 47 to 53, wherein the dose contains about 2 mg, about 4 mg, about 5 mg, about 10 mg, about 15 mg, about 20 mg, about 25 mg, or about 30 mg of plasminogen or a biologically active variant thereof.

55. The method of item 47, wherein the local administration is performed at a frequency of at least once a week, at least twice-a-week, at least every-three-day, at least every-two-day, at least once a day, or at least twice-a-day.

56. The method of item 47 or 49, wherein the medicament comprises a plurality of doses of plasminogen or a biologically active variant thereof, for performing multiple administrations until the wound size is reduced by at least 50%.

57. The method of item 47, wherein the plasminogen or biologically active variant thereof is in a solution at a concentration from about 2 mg/ml to about 20 mg/ml.

58. The method of item 57, wherein the concentration is of about 5 mg/ml, about 10 mg/ml, about 15 mg/ml or about 20 mg/ml.

59. The method of item 47, wherein the wound has an area, and wherein the dose of plasminogen or a biologically active variant thereof is proportional with said area.

60. The method of item 59, wherein the dose is expressed in mg and corresponds to about 2.5 times the wound area expressed in cm².

61. The method of item 47, wherein the wound has an area, and wherein the dose is about 10 mg when said wound area is less than 4 cm²; the dose is about 20 mg when said wound area is from 4 cm² to 8 cm², or the dose is about 30 mg when said wound area is greater than 8 cm².

62. The method of item 47, wherein the wound has a circumference, and wherein the dose of plasminogen or a biologically active variant thereof is proportional with said wound circumference.

63. The method of item 62, wherein the dose is expressed in mg and corresponds to about 1.5 times the wound circumference expressed in cm.

64. The method of any one of items 47 to 49, wherein said several injections consist of 2 to 30 injections.

65. The method of item 47, wherein the subject is a mammal, and preferably a human.

66. The method of item 47, wherein the wound is a chronic wound selected from the group of a diabetic foot ulcer, a diabetic wound, a pressure sore, a pressure ulcer, a tympanic membrane perforation, a burn wound, an incision wound, a venous leg ulcer, an arterial leg ulcer, a vascular wound or a lesion.

Further aspects of the invention will be apparent to a person skilled in the art from the following description, claims, and generalizations herein.

### BRIEF DESCRIPTION OF THE FIGURES

FIG. 1 is schematic representation of human plasminogen showing the internal bridges therein and the plasmin, u-PA and t-PA cleavage sites.

FIG. 2 represents the amino acid sequence of human plasminogen precursor as described in UniProt accession # P00747.

FIGS. 3A and 3B show photographs of the subject wound described in Example 1, before and after the plasminogen treatment, respectively.

FIGS. 4A and 4B show photographs of the subject wound described in Example 2, before and after the plasminogen treatment, respectively.

FIGS. 5A and 5B show photographs of the subject foot ulcers described in Example 3, before and after their plasminogen treatments, respectively. FIG. 5C shows a photographs of the subject described in Example 3, standing up of his feet two months after the plasminogen treatments. FIGS. 5D to 5G show photographs of the subject left foot ulcer described in Example 3 prior the treatment and at Day 8, 30, 57, 84 and 94, respectively.

FIGS. 6A, 6B and 6C show photographs of the subject right leg wound described in Example 4 prior the plasminogen treatment and at Day 33 and 56, respectively. FIGS. 6D to 6H show photographs of the subject left leg wound described in Example 4 prior the plasminogen treatment and at Day 33, 62, 93 and 129, respectively. FIG. 6I presents a graph showing the percentage of the left leg wound size observed from Day 0 to Day 129 in the subject of Example 4.

FIGS. 7A to 7D show photographs of the subject wound described in Example 5 prior the plasminogen treatment and at Day 30, 54 and 59, respectively.

FIGS. 8A, 8B and 8C show photographs of the subject wound described in Example 6 prior the plasminogen treatment and at Week 9 and 28, respectively. FIG. 8D presents a graph showing the wound size percentage observed from Day 0 to Week 28 in the subject of Example 6.

FIGS. 9A to 9E show photographs of the subject wound described in Example 7 prior the plasminogen treatment and at Week 29, 45, 64 and 88, respectively. FIG. 9F presents a graph showing the wound size percentage from the beginning of the wound (120 months prior the plasminogen treatment) until the end of the plasminogen treatment (20 months after the beginning of the plasminogen treatment) in subject described of Example 7.

FIGS. 10A to 101 show photographs of the subject wound described in Example 8 prior the plasminogen treatment and at Day 1, Day 10, Month 3, Month 5.5, Month 11, Month 12, Month 14 and Month 15, respectively. FIG. 10K presents a graph showing the wound size percentage observed from Day 0 to Month 15 in the subject of Example 8, which indicates the wound size increase resulting from the temporary interruption of in the plasminogen treatment.

### DETAILED DESCRIPTION OF THE INVENTION

Disclosed herein is a treatment or dosage regimen for promoting wound healing in a subject suffering from a wound (acute wound or chronic wound), and a medicament therefor, via treatment with plasminogen of a biologically active variant thereof.

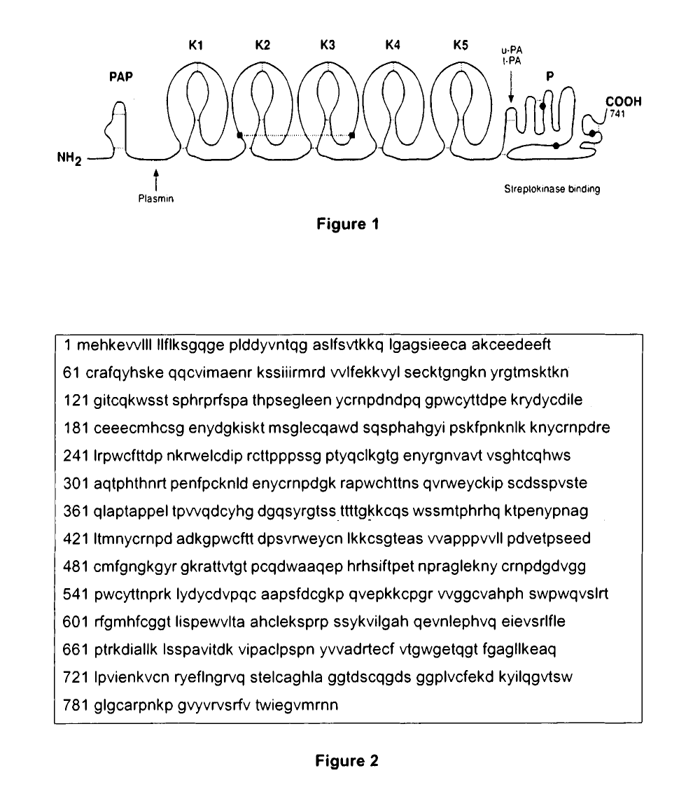

Plasminogen is a zymogen of plasmin as shown in FIG. 1. The amino acid sequence of the human plasminogen precursor is depicted in FIG. 2. Human plasminogen contains 791 amino acids (precursor=810 amino acids) with a molecular weight of about 90 kD and a pl of approximately 7.0, although differential glycosylation and/or removal of the N-terminal activation peptide can result in a isoelectric point of a range of 6.2 to 8.0. It is a single-chain protein with 24 intra-chain disulfide bridges, 5 kringle domains (involved in the binding to fibrin and to the inhibitor a2-antiplasmin), a serine protease domain (P), and an activation peptide (AP) consisting of the first 77 amino acids. There is one N-linked glycosylation site and one O-linked site, although a second O-linked site has been identified (Goldberg, 2006). Approximately 70% of the Plasminogen in circulation contains only O-linked glycosylation while the rest contains both N- and O-linked sugars.

Native Plasminogen is produced in two main forms, Glu-Plasminogen (Glu-Pg) and Lys-Plasminogen (Lys-Pg), named for the N-terminal amino acid of either glutamic acid or lysine, respectively. Glu-Pg is composed of the entire amino acid sequence designated by the gene sequence (excluding the activation peptide), while Lys-Pg is the result of a cleavage of the Glu-Pg between Lys-77 and Lys-78 (underlined in FIG. 2). The circulating half-life of Lys-Pg is considerably shorter than Glu-Pg (2-2.5 days for Glu-Pg, 0.8 days for Lys-Pg). Glu-Pg is the dominant form of Pg present in plasma with very little Lys-Pg detected in the circulation (Violand, B. N., Byrne, R., Castellino F. J. (1978) The effect of α-, ω-Amino Acids on Human Plasminogen Structure and Activation. J Biol Chem. 253 (10): 5395-5401; Collen D, Ong E B, Johnson A J. (1975) Human Plasminogen: In Vitro and In Vivo Evidence for the Biological Integrity of NH₂-Terminal Glutamic Acid Plasminogen. Thrombosis Research. 7:515-529).

Plasminogen is synthesized in the liver and secreted into plasma. Plasminogen is distributed throughout the body and when conditions are present for activation, the Plasminogen pro-enzyme is converted to the active enzyme, plasmin, by tissue-type plasminogen activator (t-PA) or by urokinase plasminogen activator (u-PA). Plasmin then degrades fibrin and converts latent matrix metalloproteinases (pro-MMPs) into active MMPs, which in turn further degrade extracellular matrix (ECM) as part of the tissue healing/remodeling process. Plasminogen activation mediated by t-PA is primarily involved in fibrin homeostasis, while plasmin generation via u-PA, forming a complex with its receptor u-PAR, plays a role in tissue remodeling.

In subjects suffering from diabetes, plasminogen is glycosylated in the blood circulation. Glycosylated plasminogen is non-functioning or mal-functioning. As a result, a wound in a diabetes subject has more difficulties to heal by itself and is prone to not heal and become a chronic wound. In diabetes subject, the local administration of plasminogen to treat a wound is an advantage considering that an intravenously administered plasminogen may get glycosylated prior to reach the wound. This is particularly a problem in diabetes subjects where the blood glucose is not controlled or partly controlled.

In the surrounding area of a wound area, the tissues may be damaged. Hypoxia, ischemia, infection and/or swelling are often present. These conditions interfere with the normal blood circulation accessing the wound and can represent an obstacle for an intravenously administered compound to reach the wound. Therefore, the local administration of a drug to a wound is an advantage in order to enable the compound to access more rapidly and more efficiently the wound and the cells of the disrupted tissues.

In an aspect of the invention, the local administration is intracutaneous (or intradermal), subcutaneous (or subdermal), intramuscular or topical. In an aspect of the invention, said administration is performed in the wound, in the vicinity of the wound or around the wound. Preferably, the administration is performed outside the wound but in close proximity thereto. When the administration is intracutaneous (or intradermal), subcutaneous (or subdermal) or intramuscular, the dose can be administered one injection (single injection) or in several injections (multiple injections). Preferably, the local administration consists of more than one injection. The number of injections may also vary in relation with the size of the wound, i.e. the length of the wound periphery. In a preferred embodiment, the number of injections is between 2 and 30 injections, or between 3 and 12 injections. In an embodiment of the present invention, the injection(s) is(are) performed at a distance of about 0.1 cm to about 2 cm, and preferably at a distance of about 0.1 cm to about 1 cm from the wound. In an embodiment, the injection(s) is(are) performed at a distance of about 0.5 cm or about 1 cm from the wound. In condition where a wound that is not an opened wound (such as bedsore of stage 1, or burn when the dermis remains continuous, or lesion), the local administration is preferably performed in the wound area. In condition where a wound that is an opened wound (such as cut, ulcer or the like), the local administration is preferably performed around the wound area. When the administration is topical, the dose of plasminogen can be administered by means of a drop, a cream, a gel or the like. The topical administration via the use of drops is preferred when the wound is a tympanic membrane perforation, or when the wound is in the eye such as a ligneous conjunctivitis in the eye lid. Nonetheless tympanic membrane perforation and ligneous conjunctivitis can also be treated by administering plasminogen intracutaneously or subcutaneously around the tympanic membrane perforation, or in the eye lid in the case of a ligneous conjunctivitis. The ligneous conjunctivitis wound is usually referred to as a lesion. In embodiments where plasminogen is contained in a drop solution, a cream, a gel or the like, the topical administration can be either applied on the wound, around the wound, or both.

In an aspect, the treatment described herein comprises the repeated administration of a dose of plasminogen or a biologically active variant thereof of about 0.4 mg to about 30 mg. According to embodiments of the invention, the administration is twice-a-day, daily or more often, every-two-day or more often, every-three-day or more often, twice-a-week or more often, every-four-day or more often every-five-day or more often, or weekly or more often.

The term “Plasminogen” as used herein refers to any form of a native plasminogen polypeptide (e.g., Glu-plasminogen or Lys-plasminogen) from any animal, for example a mammal (e.g., human). Plasminogen is a pro-enzyme that is converted to the active enzyme, plasmin, by tissue-type plasminogen activator (t-PA) or by urokinase plasminogen activator (u-PA). Plasmin then degrades fibrin and converts latent matrix metalloproteinases (pro-MMPs) into active MMPs, which in turn further degrade extracellular matrix (ECM) as part of the tissue healing/remodeling process. The term “biologically active variant” as used herein refers to an altered plasminogen polypeptide that retains the biological activity of native plasminogen, i.e. the ability to be converted to a plasmin polypeptide (by t-PA and or u-PA) that is able to degrade fibrin and converts latent matrix metalloproteinases (pro-MMPs) into active MMPs. The variant may comprises one or more amino acid substitutions, deletions/truncations (N-terminal, C-terminal, and/or internal amino acid deletions/truncations), additions (N-terminal, C-terminal, and/or internal amino acid additions). The biologically active variants may exhibit a biological activity (e.g., enzymatic activity of the resulting plasmin) that is lower, higher or similar to that of a native plasminogen polypeptide. In embodiments, the variant has at least 60, 70, 75, 80, 85, 90, or 95% amino acid sequence identity with a native plasminogen polypeptide. Biologically active plasminogen variants are described, for example, in WO2012/093132, WO2013/024074 and in Wang et al. (1995, Protein Science 4, 1758-1767), and included the truncated variants of plasminogen commonly referred to as “midiplasminogen”, “miniplasminogen”, “microplasminogen” and “delta-plasminogen” that lack one or more kringle domains and/or parts thereof. In an embodiment, the plasminogen is human plasminogen. In a further embodiment, the composition comprises native human plasminogen. The plasminogen may be obtained from several sources. It may be obtained by recombinant synthesis, or extracted/purified from blood, plasma or a blood-derived solution. Plasminogen can be extracted from blood or plasma for example by Cohn fractionation or by precipitation. Plasminogen can be purified from plasma or blood-derived solution by for example binding affinity chromatography, such as the method described in WO 2006/120423, or produced recombinantly.

The term “pharmaceutical composition” and “medicament” are interchangeably used herein. The medicament necessarily includes a pharmaceutical composition of plasminogen or a variant thereof. In certain aspect of the invention, the medicament further includes a device for administering the pharmaceutical composition. Said composition can be in the form of a powder or in the form of a solution. In the embodiment where the composition is in a powder form, it is preferably ready for reconstitution prior to injection or local administration.

In an embodiment of the present invention, the plasminogen or biologically active variant thereof contained in the pharmaceutical composition has a purity of about 70% or more, of about 80% or more, of about 85% or more, of about 90% or more, of about 95% or more, and of about 98% or more. In another embodiment of the present invention, the plasminogen or biologically active variant thereof is contained in a pharmaceutical composition where the total amount of protein other than the plasminogen or variant thereof is less than about 30%, less than about 20%, less than about 15%, less than about 10%, less than about 5%, or less than about 2%.

In an embodiment of the present invention, the pharmaceutical comprising the plasminogen or its variant, does not comprise or is substantially free of, an additional protein (i.e. in addition to the plasminogen or variant thereof). In an embodiment of the present invention, the composition does not comprise or is substantially free of albumin. In an embodiment of the present invention, the composition or medicament does not comprise or is substantially free of aprotinin or no more than 10 KIU/mL of aprotinin. In an embodiment of the present invention, the composition or medicament does not comprise or is substantially free of bovine aprotinin or no more than 10 KIU/mL of bovine aprotinin. In an embodiment of the present invention, the composition or medicament does not comprise or is substantially free of synthetic aprotinin or no more than 10 KIU/mL of synthetic aprotinin. In an embodiment of the present invention, the composition does not comprise or is substantially free of a trypsin inhibitor. In an embodiment of the present invention, the composition does not comprise or is substantially free of a serine protease inhibitor. In an embodiment of the present invention, the composition does not comprise or is substantially free of plasmin. In an embodiment of the present invention, the composition is substantially free or free of a surfactant, i.e. the concentration of surfactant is less than 0.01 mM.

The term “subcutaneous” as used herein refers to an administration or a delivery beneath the skin or into the hypoderm, and is equivalent to the term “subdermal”. The term “intracutaneous” as used herein refers to an administration or a delivery within the skin, and is equivalent to the term “intradermal”. The term “intramuscular” as used herein refers to an administration or a delivery in the muscle tissue. The term “topical” as used herein refers to an administration or a delivery on the skin or on the wound.

The term “daily” as used herein refers to the administration of a dose of plasminogen or a biologically active variant thereof once a day. Preferably, the administration is performed at about the same time of the day, although not necessary. Occasionally, the administration may be omitted one or two days during the treatment period without departing from the intended meaning of a daily administration. The term “occasionally” in this context means once or twice a week.

The term “promoting” as used herein refers to stimulation, acceleration, and/or improvement. The term “healing” as used herein refers to recovery, repair, treatment, or restoration. The term “wound” as used herein includes an opened wound and a non-opened wound. An opened wound refers to an opening in the skin, including without limitation, a lesion, an incision, a laceration, a cut, an ulcer, a damage, or any disruption of the skin integrity. A non-opened wound refers to a wound where the skin is not opened but the skin tissue has an abnormal color, elevated temperature and/or abnormal firmness (either firmer or softer than the area around it), and the tissues underneath the skin are characterized by inflammation, higher sensitivity than normal, and/or swelling.

It has been found that a repeated dose as low as 2 mg per day is sufficient to promote wound healing. Acceleration of the wound healing may be increased by raising the dose up between 3 mg and 30 mg, and preferably at 4 mg, 5 mg, or 10 mg. Various doses of plasminogen have been administered in the examples, and it has been noted that above a certain quantity, the mechanisms of repair appear saturated and the wound does not heal faster. Therefore, in an embodiment, the range of plasminogen or a biologically active variant thereof to be administered per dose is about 2 mg to about 30 mg. In another embodiment, the range for the dose of plasminogen or a biologically active variant thereof to be administered per dose is about 2 mg to about 20 mg. In further embodiments, the dose is about 2 mg, about 4 mg, about 5 mg, about 10 mg, about 15 mg, about 20 mg, about 25 mg or about 30 mg. In another embodiment, the dose of plasminogen or a biologically active variant thereof is about 10 mg.

The frequency of administration varies from twice-a-day to once-a-week, preferably from once-a-day to once-a-week, and more preferably at once-a-day, every-two-day, every-three day, every two or three days, twice-a-week, or once-a-week. In an embodiment, the frequency of administration is every two or three days. In an embodiment, the frequency of administration is once-a-day.

In an embodiment, the dose of plasminogen or a biologically active variant thereof is determined in relation with the wound size (area) using the following equation:

X=(m)Y

wherein:

X=dose of plasminogen or a biologically active variant thereof (mg);

Y=wound area (cm²); and

m=2.0 to 3.0, in an embodiment 2.2 to 2.8, in a further embodiment 2.4 to 2.6.

In an embodiment, m=2.5 and the equation is:

X=(2.5)Y

In another aspect, the present invention provides a method for treating a wound in a subject, the method comprising (i) determining the size of the wound; (ii) determining the dose of plasminogen or a biologically active variant thereof to be administered to the subject using one of the above equations; and locally administering said dose of plasminogen or a biologically active variant thereof at a frequency of twice-a-day to once-a-week.

In an embodiment, the determination of the dose of plasminogen or a biologically active variant thereof is based on the initial wound area, i.e. the wound area measured before the treatment. The ‘treatment’ designates the administration (e.g., multiple or repeated administrations) of a dose of plasminogen or a biologically active variant thereof. In an embodiment, the dose of plasminogen or a biologically active variant thereof administered remains the same throughout the duration of the treatment, i.e. for all repeated administrations. In another embodiment, the dose of plasminogen or a biologically active variant thereof is re-determined in course of the treatment in function of a ‘wound area in treatment’. As the size of the wound area is expected to decrease during the treatment, there is an embodiment where the dose of plasminogen or a biologically active variant thereof administered is lowered during the course of the treatment in relation with the wound area reduction, and which may be determined using the above-noted equations. The re-evaluation of the dose can be performed once during the treatment or several times during the treatment. There is another embodiment where the dose of plasminogen or a biologically active variant thereof administered is increased during the course of the treatment in order to accelerate the wound healing.

In another embodiment, the dose of plasminogen or a biologically active variant thereof can be determined in relation with the wound size (area) by using the following chart:

| Wound area | | Dose |
|---|---|---|
| <4 | cm² | about 10 mg |
| 4-8 | cm² | about 20 mg |
| >8 | cm² | about 30 mg |

In an embodiment, the dose of plasminogen or a biologically active variant thereof can be determined in relation with the wound circumference using the following equation:

X=(n)Z

wherein:

X=dose of plasminogen or a biologically active variant thereof (mg),

Z=wound circumference (cm), and

n=1.0 to 2.0, in an embodiment 1.2 to 1.8, in a further embodiment 1.4 to 1.6.

In an embodiment, n=1.5 and the equation is:

X=(1.5)Z

In an embodiment, the frequency of administration of a dose of plasminogen or a biologically active variant thereof (each dose) may vary in relation with the wound size (area). For instance, the doses may be administered less frequently when the wound size is reduced by more than 50%. For example, the initial frequency of administration can be daily, and when the wound size is reduced by more than 50% of its initial size, the frequency can be reduced to every-two-day. The frequency can be further reduced to once-a-week. The frequency may be re-evaluated once or several times during the treatment.

Optimally, the “duration of the treatment” starts when the subject is identified as a subject in need of receiving a treatment for promoting the wound healing and ends when the wound is closed. However, for various reasons, the treatment may not start right away when the subject in need thereof is identified. For various reasons, the treatment may stop before the wound is closed. However, in some cases (such as in patients having an impaired mechanism of repair and in plasminogen-deficient patients), it may be desirable to pursue the treatment until the wound is closed. According to an embodiment of the invention, the treatment stops when the wound starts to heal by itself and the size of the opening starts to reduce. It may be desirable to extend the duration of treatment beyond the wound closure in some cases, in order to assure a definitive healing. Preferably, the duration of treatment is at least 3 days, at least 11 days, at least 14 days, at least 18 days, at least 21 days, at least 22 days, at least 28 days, at least 30 days, at least 32 days, or at least 52 days. In a preferred embodiment, the duration of treatment is at least 21 days. In another preferred embodiment, the duration of treatment ends when significant improvements of the wound conditions are observed, such as redness reduction and/or significant wound size reduction. A significant wound size reduction is defined as, a wound size of 75%, 50%, 40%, 30%, 20%, or 10% compared to the initial wound size (before treatment). In an embodiment, the duration of treatment ends when the wound size is reduced by at least 50%, at least 75%, at least 90% or by 100%. A reduction of the wound size of 100% means that the wound is completely healed. A complete healing means that the wound is closed in the case of an opened wound, or disappeared in the case of non-opened wound, such a lesion for example.

In an embodiment, the dose of plasminogen or a biologically active variant thereof is in a pharmaceutical composition. Said composition can be in the form of a powder or a liquid solution. The plasminogen or a biologically active variant thereof contained in said solution is preferably at a concentration of about 1 mg/ml to about 20 mg/ml, and more preferably at a concentration of about 1 mg/ml, about 5 mg/ml, about 10 mg/ml, about 15 mg/ml or about 20 mg/ml. In a preferred embodiment, the amount of plasminogen or a biologically active variant thereof contained in said solution is in a concentration of about 10 mg/ml.

The wound that is treated using the methods, uses and medicaments of the present invention may be an acute wound or a chronic wound. A chronic wound is intended to define a wound that has remained unhealed or opened for a long time, or that appears to not heal for a period of at least three months, or that remains opened for at least three months, or that does not show the characteristic of healing, or that shows the presence of pus, putrefaction or gangrene. An acute wound is intended to define a wound that is not a chronic wound. Examples of wounds that are intended to be treated with the methods, uses and medicaments of the present invention, includes, without limitation, a diabetic foot ulcer, a diabetic wound, a pressure sore, pressure ulcer, vascular wound, venous leg ulcer, arterial leg ulcer, a tympanic membrane perforation, a burn wound, an incision wound, a lesion, a ligneous conjunctivitis wound.

In a preferred embodiment, the wound is a topical or mucosal wound, or a wound of the epidermis, the dermis or the mucous membrane of a subject. In an embodiment, the subject is a mammal. The term mammal includes an animal or a human.

In an embodiment, the medicament of the present invention contains all the doses of plasminogen or a biologically active variant thereof that are necessary for the whole duration of the treatment. In another embodiment, the dosage form of the medicament is for one-administration use and contains one dose of plasminogen or a biologically active variant thereof. In this embodiment, the device to inject the plasminogen or the biologically active variant thereof can be disposable. In the embodiment where the dosage form of the medicament is for one-use, the dose of plasminogen or a biologically active variant thereof for one administration is preferably already inserted into said device. For example, the medicament can be in the form of a disposable syringe containing a single dose (i.e. between about 2 mg to about 30 mg plasminogen or a biologically active variant thereof). In another example, the medicament can be in the form of several pre-filled disposable syringes where each of them contains a single dose (i.e. between about 2 mg to about 30 mg plasminogen or a biologically active variant thereof). In accordance with an embodiment of the present invention, the medicament includes preferably several pre-filled disposable syringes so as to cover to the whole duration of the treatment or a part of the duration of the treatment, and preferably at least 21 pre-filled disposable syringes or at least 28 pre-filled disposable syringes. When the medicament includes medicament covering the doses necessary for covering a part of the duration of the treatment, said medicament can be renewed until the wound is completely healed or until the wound size is reduced by more than 50% of its initial size. According to another embodiment, the medicament is in the form of a one container that contains multiple doses therein. According to a further embodiment, the medicament further includes at least 21 disposable syringes so as to be able to administer the plasminogen or the biologically active variant thereof for the whole duration of the treatment or for a part of the whole duration of the treatment. In another embodiment, the syringe(s) (disposable or not) is(are) provided by the doctor, the nurse, or purchased separately from the medicament. In an embodiment, the device is a pen adapted for multiple injections. In another embodiment, the device is a needle-less pen adapted for multiple injections. Said syringe (disposable or not), said needles, said pen and said needle-less pen are adapted for intracutaneous, subcutaneous or intramuscular delivery.

According to another embodiment, the medicament is in the form of a one container that contains multiple doses of plasminogen or a biologically active variant thereof therein, wherein said doses are in the form of a solution or a powder which is ready for reconstitution into a solution. Said powder can be the result of a lyophilisation process or obtained other means.

The term “pharmaceutical composition” designates a composition for pharmaceutical, medical or therapeutic purposes. Pharmaceutical compositions generally comprise One or more pharmaceutically acceptable carriers or excipients, and may be prepared in a manner well known in the pharmaceutical art by mixing the active ingredient having the desired degree of purity with one or more optional pharmaceutically acceptable carriers, excipients and/or stabilizers. Supplementary active compounds can also be incorporated into the compositions. The carrier/excipient can be suitable, for example, for subcutaneous or intradermal administration (see Remington: The Science and Practice of Pharmacy by Alfonso R. Gennaro, 2003, 21^{th} edition, Mack Publishing Company).

An “excipient,” as used herein, has its normal meaning in the art and is any ingredient of an intracutaneous, subcutaneous, intradermal or topical dosage form that is not an active ingredient (drug) itself. Excipients include for example a tonicity modifier, a stabilising agent, a bulking agent, a preservative, or the like. The stabilising agent can comprise (i) an amino acid, (ii) an amino acid salt, (ii) an amino acid analog, or (iv) any mixture thereof. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects.

The plasminogen that is formulated in the composition of the present invention may be obtained from several sources. It may for example be obtained by recombinant synthesis, or extracted/purified from blood, plasma or a blood-derived solution. Plasminogen can be extracted from blood or plasma by Cohn fractionation or by precipitation. Plasminogen can be purified from plasma or blood-derived solution by a binding affinity chromatography, such as the method described in WO 2006/120423. A variant of plasminogen includes, without limitation, any modifications of the amino acid sequence or any additions of a group thereto or any additions of an amino acid or an amino acid sequence thereto. A fragment of plasminogen includes, without limitation, any deletions of an amino acid or an amino acid sequence thereto as long as the plasminogen activity is maintained or partly maintained.

As used herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

The term “subject” includes living organisms which can benefit from an administration of plasminogen, a variant thereof or a fragment thereof. The term “subject” includes animals such as mammals, pets or birds. Preferably, the subject is a mammal or a human. More preferably, the subject is a human. Most preferably, the subject is a human patient having a wound, including an acute wound or a chronic wound. Also most preferably, the subject is a human patient having a chronic wound. In an embodiment, the mammal is a higher mammal. Examples of higher mammals include, without limitation, non-human primate, horse, cow, elephant. Examples of pets include, without limitation, dogs, cats, rabbits, horses, ponies.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

The terms “treatment” or “treating” of a subject includes the application or administration of a composition of the invention to a subject (or application or administration of the composition of the invention to a tissue or an organ of a subject) with the purpose of delaying, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In some embodiments, the term “treating” can include increasing a subject's life expectancy and/or delay before additional treatments are required.

As used herein, the term “therapeutically effective amount” means the amount of compound that, when administered to a subject for treating or preventing a particular disorder, disease or condition, is sufficient to effect such treatment or prevention of that disorder, disease or condition. As used herein, the term “therapeutically effective amount” further means the amount of plasminogen, its variant or its fragment that is effective to heal a wound.

According to the present invention, the dose (or amount) of plasminogen or biologically active variant thereof is administered subcutaneously, intracutaneously or intramuscularly. In an embodiment, said dose or amount is administered subcutaneously. In another embodiment, said dose or amount is administered intracutaneously. As used herein, the term “intracutaneously’ and “intradermal” mean the same and be used interchangeably. In another embodiment, said dose or amount is administered intramuscularly.

It should be noted that the term “formulation” and “composition” and “preparation” may be used herein alternatively and are intended to designate the same.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

### EXAMPLES

The following examples further illustrate the practice of this invention but are not intended to be limiting thereof. In all the following examples, the injections were performed with a syringe of 1 ml having a needle of 0.3 mm for intradermal or subcutaneous administration. The plasminogen dose was distributed in several injections around the wound, at about 0.5 cm from the wound.

### Example 1: Burn Wound Healing

A 34-year old healthy woman with burn injuries on both legs has received 7 times skin tissue expansion over a period of 10 years. Without any healing progress with known conventional treatment, the wound in the distal area of the right knee is considered a chronic wound.

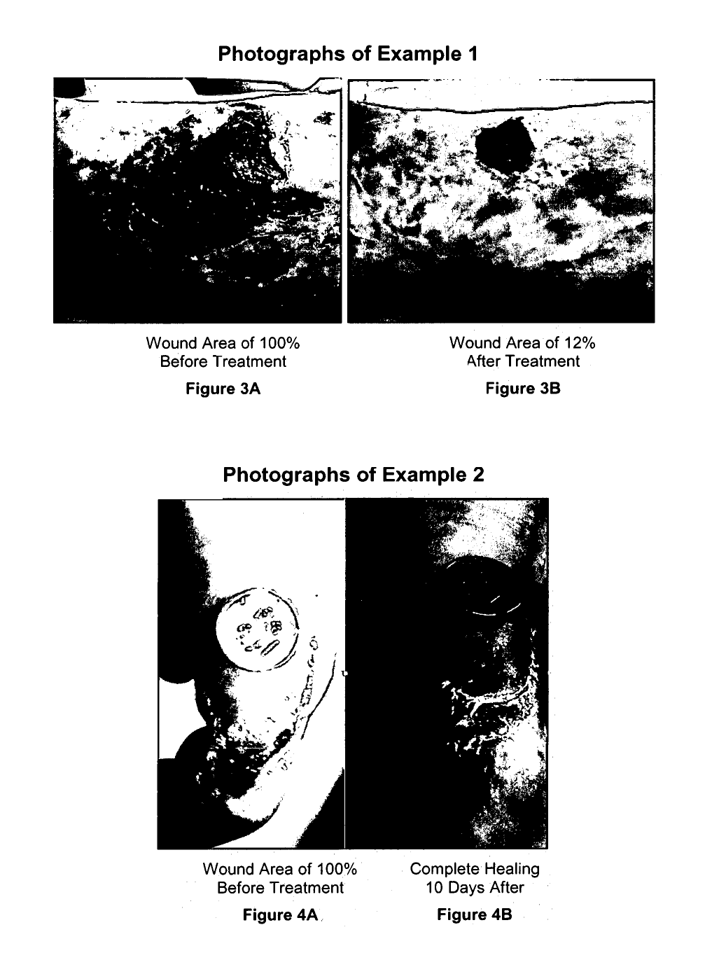

Plasminogen treatment consisted of 22 doses of 4 mg of plasminogen during a period of 9 months. After the 9-month treatment, the wound size was decreased by 88% of its initial size. Photographs of the wound prior and after the plasminogen treatment are shown in FIGS. 3A and 3B, respectively.

### Example 2: Cut Wound Healing

A 62-year old man had a wound caused by a saw on the lateral side of his right hand. The wound was treated with revision and suturing, but nonetheless a deep infection has taken place. After debridement, the wound was treated with traditional local treatment without any improvement during 2 months.

Plasminogen treatment consisted of a 3-day treatment of 4 mg of plasminogen daily. The wound was completely healed 10 days after the beginning of the treatment, and the man went back to work. Photographs of the wound prior the plasminogen treatment and 10 days after the beginning of the treatment are shown in FIGS. 4A and 4B, respectively.

### Example 3: Foot Ulcer Healing

A non-diabetic 77-year old man, with artherosclerosis and severe foot neuropathy, was suffering from a chronic foot ulcer on each foot during 3 years. Distal, partial amputation was performed 1.5 year before the plasminogen treatment on the first toe of the left foot. Antibiotic treatments have been administered but the wound has remained opened. The patient was arrived at a point where a bilateral below-the-knee amputation was evaluated as a solution for removing patient's pain.

Plasminogen treatment of the left foot ulcer consisted of 21 plasminogen doses during a period of 56 days. Plasminogen treatment of the right foot ulcer consisted of 32 plasminogen doses during a period of 84 days. The initial 8th doses contained 2 mg of plasminogen and were injected once a day or every-two day. The latter doses contained 5 mg of plasminogen and were injected every-two-day or every-three-day. After the plasminogen treatments, the foot ulcers were completely healed and the patient could walk and balance on the front of his feet. No side effect of the plasminogen treatment was observed or reported by the patient.

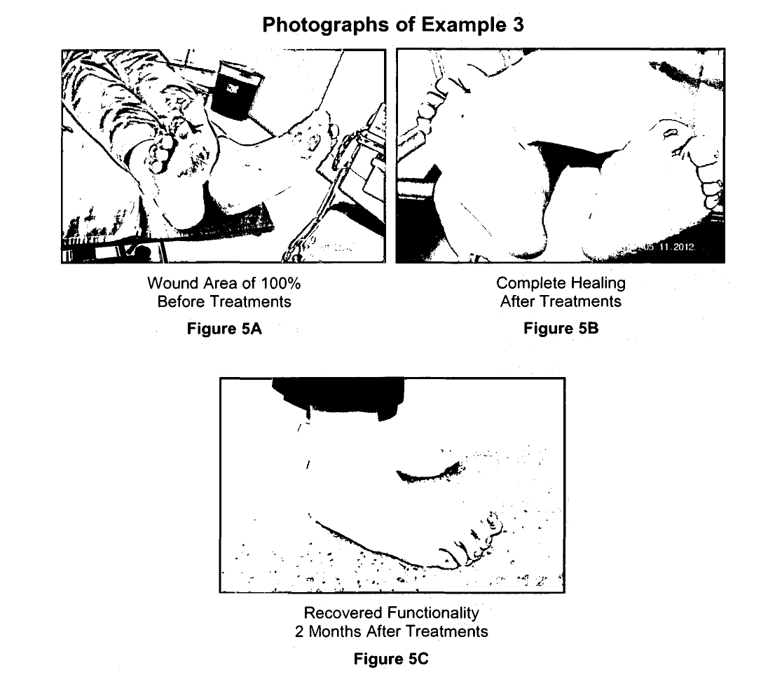

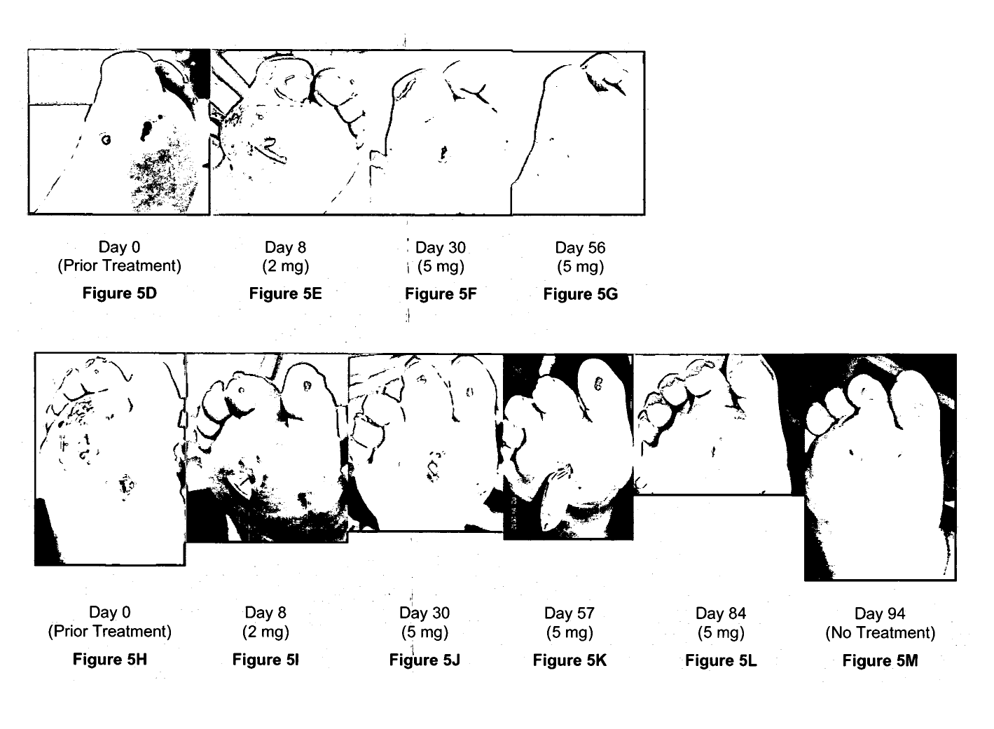

Photographs of the foot ulcers prior the plasminogen treatments and after the treatments are shown in FIGS. 5A and 5B, respectively. FIG. 5C shows the feet of the patient when standing up, about 2 months after the end of the treatment. FIGS. 5D, 5E, 5F and 5G show the left foot at Day 0 (prior treatment), at Day 8 (2 mg), Day 30 (5 mg) and Day 56 (5 mg) of plasminogen treatment, respectively. FIGS. 5H, 5I, 5J, 5K, 5L and 5M show the right foot at Day 0 (prior treatment), at Day 8 (2 mg), Day 30 (5 mg), Day 57 (5 mg), Day 84 (5 mg) and Day 94 (no treatment) of the plasminogen treatment, respectively.

### Example 4: Postoperative Wound Healing

A 80-year old woman suffering from arteriosclerosis had vein grafts harvested from her legs in order to perform a coronary bypass. Three months after the bypass, she was left with unhealed postoperative wounds in her left and right legs. The wounds were treated with painful debridements without improvement. The patient was suffering from bilateral neuropathy in both legs.

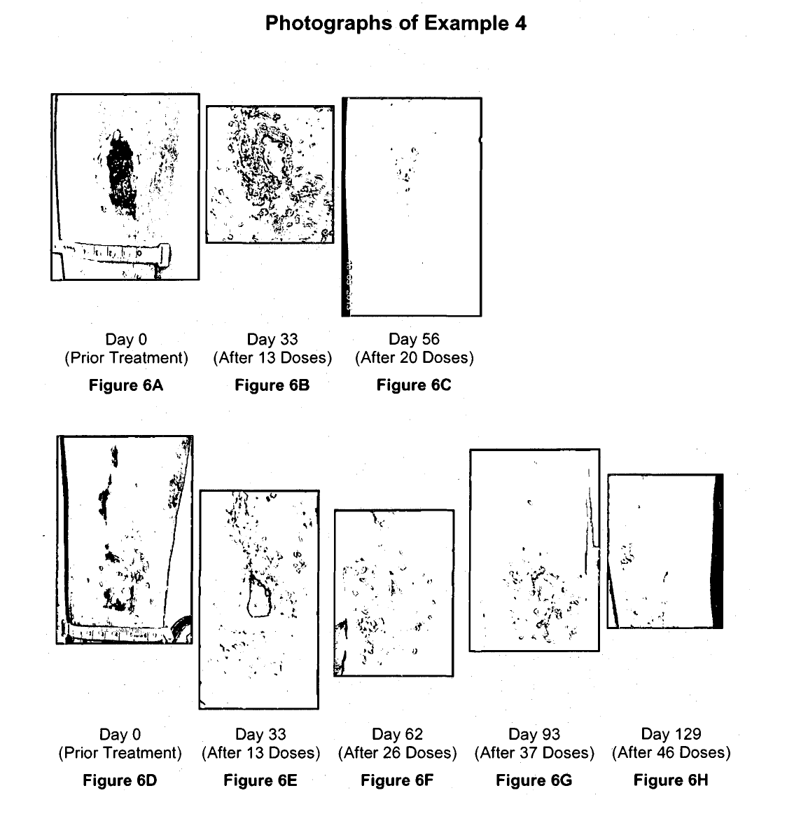

Plasminogen treatment of the right leg wound consisted of 20 doses of 5 mg of plasminogen during a period of 56 days, wherein the first 13 doses were injected within the first 33 days of treatment. The administration of plasminogen was performed at every two or three days. Complete healing of the right leg wound was observed after 56 days of treatment. Photographs right leg wound at Day 0 (prior treatment), Day 33 and Day 56 of the plasminogen treatment are shown in FIGS. 6A, 6B and 6C, respectively.

Plasminogen treatment of the left leg wound consisted of 46 doses of 5 mg of plasminogen during a period of 129 days, wherein the first 13 doses were injected within the first 33 days of treatment, the following 13 doses were injected during a period of 29 days, the subsequent 11 doses were injected during another period of 31 days, and the later 9 doses were injected within the last period of 36 days. The plasminogen administration frequency was initially at every two or three days, and was extended up to every four days. Complete healing of the left leg wound was observed after 129 days of treatment. Photographs left leg wound at Day 0 (prior treatment), Day 33, Day 62, Day 93 and Day 129 of plasminogen treatment are shown in FIGS. 6D, 6E, 6F, 6G and 6H, respectively. Left leg wound size percentages from Day 0 to Day 129 are reported in the graph shown in FIG. 6I.

### Example 5: Trauma Wound Treatment

A 40-year old man with insulin-dependent type 1 diabetes mellitus, was injured by a cow that had stepped on his right foot. For two years, the wound had been treated with different processes: including an immobilizing boot, a partial and distal amputation of a toe, a deep infection drainage, intravenous antibiotic treatments and immobilization.

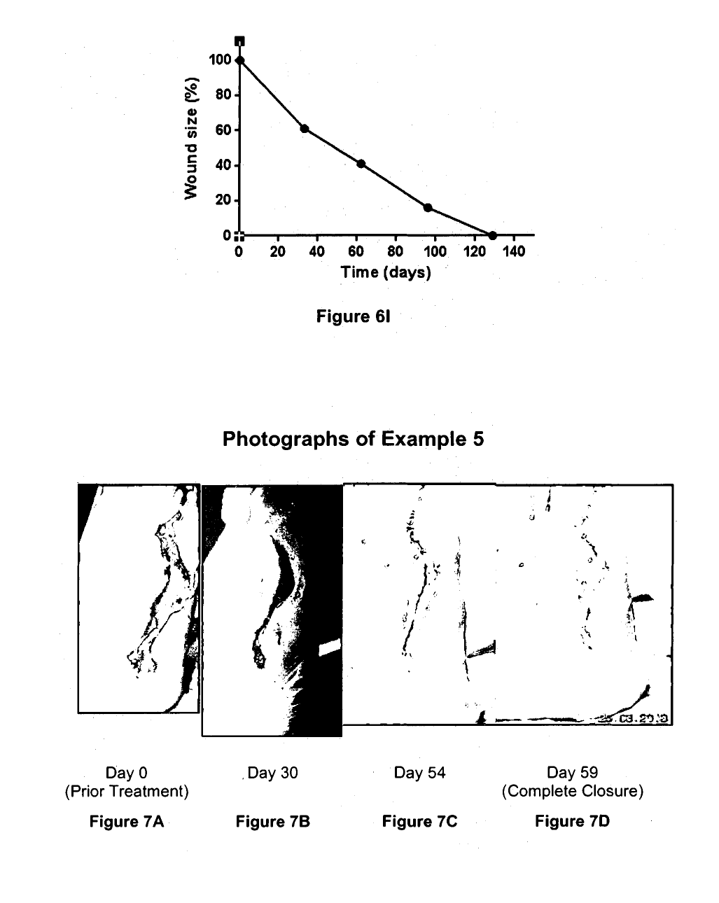

Plasminogen treatment consisted of 23 doses of 5 mg of plasminogen during a period of 52 days, wherein the first 10 doses were injected within the first 24 days of treatment. The administration of plasminogen was performed at every two or three days. At the beginning of the treatment, a reduction of the bad smell associated with the wound has been observed. At Day 24, the patient has started to work again. At Day 59, the wound was completely closed as shown in FIG. 7D.

Photographs of the wound at Day 0 (prior treatment), Day 30, Day 54 and Day 59 of plasminogen treatment are shown in FIGS. 7A, 7B, 7C and 7D, respectively.

### Example 6: Chronic Wound Treatment

A 58-year old man with a chronic wound underneath his left foot, which was caused by walking on a piece of glass 15 years ago. Many antibiotic treatments were administered, which were not efficient to heal the wound. ement. During these 15 years, the man had difficulties to use his foot.

Plasminogen treatment consisted of a dose of 5 mg of plasminogen once a week for a period of 28 weeks. After 28 weeks, swelling of the tissue was reduced and the wound size has decreased by 32% of its initial size. No negative side effect was observed during and after each dose administration.

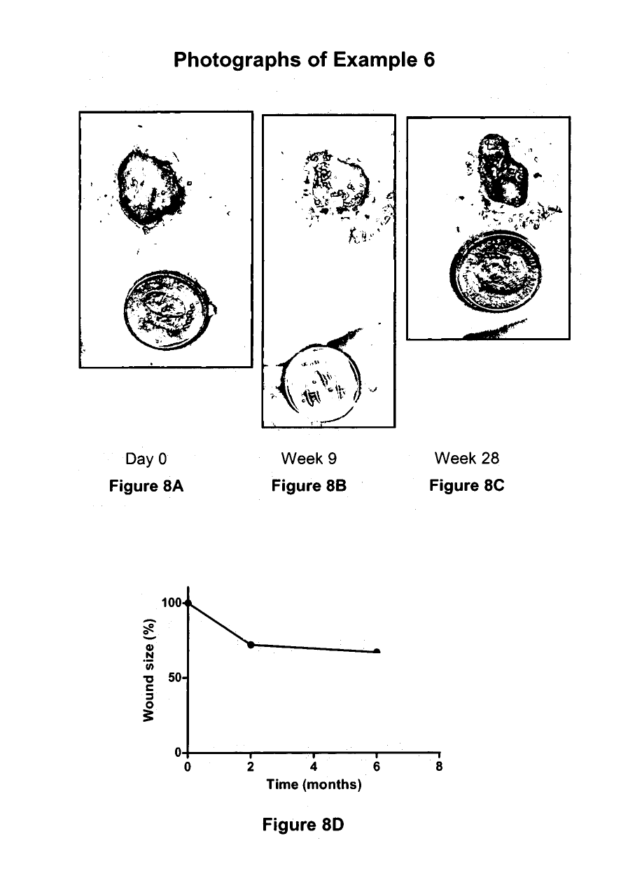

Photographs of the wound at Day 0 (prior treatment), Week 9 and Week 28 of plasminogen treatment are shown in FIGS. 8A, 8B and 8C, respectively. Wound size percentages at Day 0 (100%), Week 9 (74%) and Week 28 (62%) are reported in the graph shown in FIG. 8D.

### Example 7: Chronic Wound Healing

A 66-year old woman had a chronic wound for 9 years on the lateral malleal region of her left foot. The patient underwent multiple known conventional wound treatments without improvement.

Plasminogen treatment consisted of repeated doses of 10 mg of plasminogen. For the first two weeks, doses were administered daily, then 3 times a week, and later once a week. After 20 months of treatment, the wound size has reduced by 91% of its initial size, and the patient's vitality and social life were significantly improved.

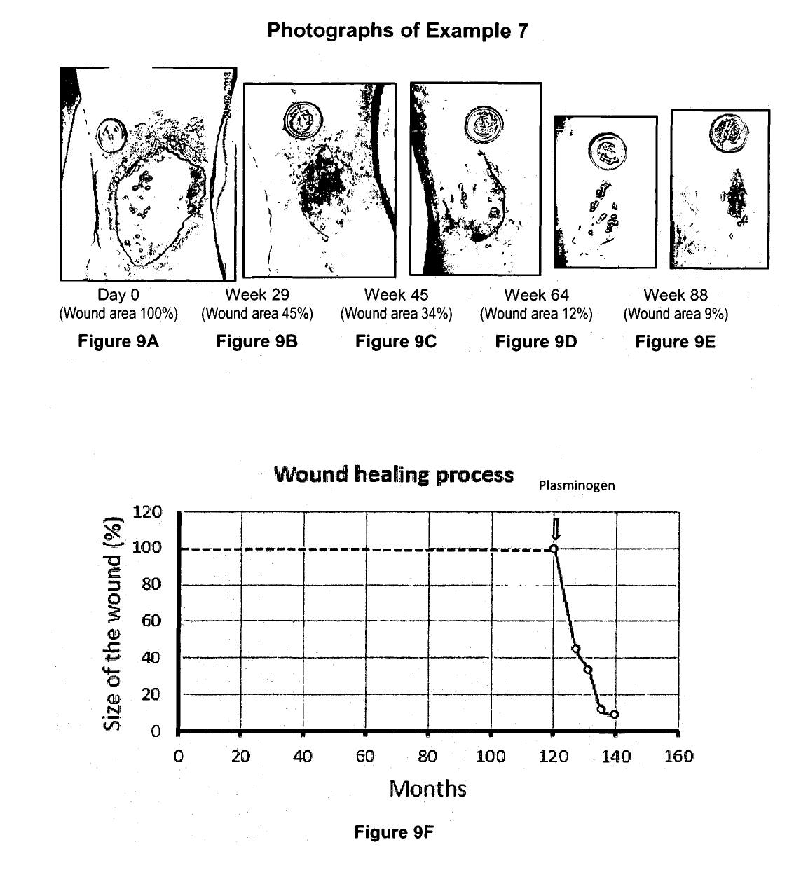

Photographs of the wound at Day 0 (prior treatment), Week 29, Week 45, Week 64 and Week 88 of plasminogen treatment are shown in FIGS. 9A, 9B, 9C, 9D and 9E, respectively. A graph reporting the wound size percentage during conventional wound treatments and the plasminogen treatment is shown at FIG. 9F.

### Example 8: Chronic Wound Healing

A 71-year old man had a wound located to the Achilles heel region for 3 years. The tendon had a vasculitis. The patient was also affected by several chronic conditions such as goit, arteriosclerosis, hypertonia and obesity, but the patient was not diabetic. The wound had been treated with multiple known conventional interventions including intravenous antibiotics treatments with Enibrel™. The Enibrel™ treatments were stopped after 3 cycles due to lack of any improvement. The wound was severely infected. An immunosuppressive treatment was further administered since the cause of the wound was suspected to be a vascular genesis.

Plasminogen treatment consisted of daily doses of 10 mg of plasminogen for a period of 14 days. After that period, the conditions of wound have improved and the plasminogen treatment was stopped for a period 14 days. This interruption of plasminogen treatment has resulted in the worsening the wound, which has returned to its initial state. Thus, plasminogen treatment has been restarted at the same regimen i.e. daily doses of 10 mg. After few months of plasminogen treatment, the wound conditions were significantly improved and the immunosuppressive medicine was stopped. After 15 months of treatment, the wound size has reduced by 60%.

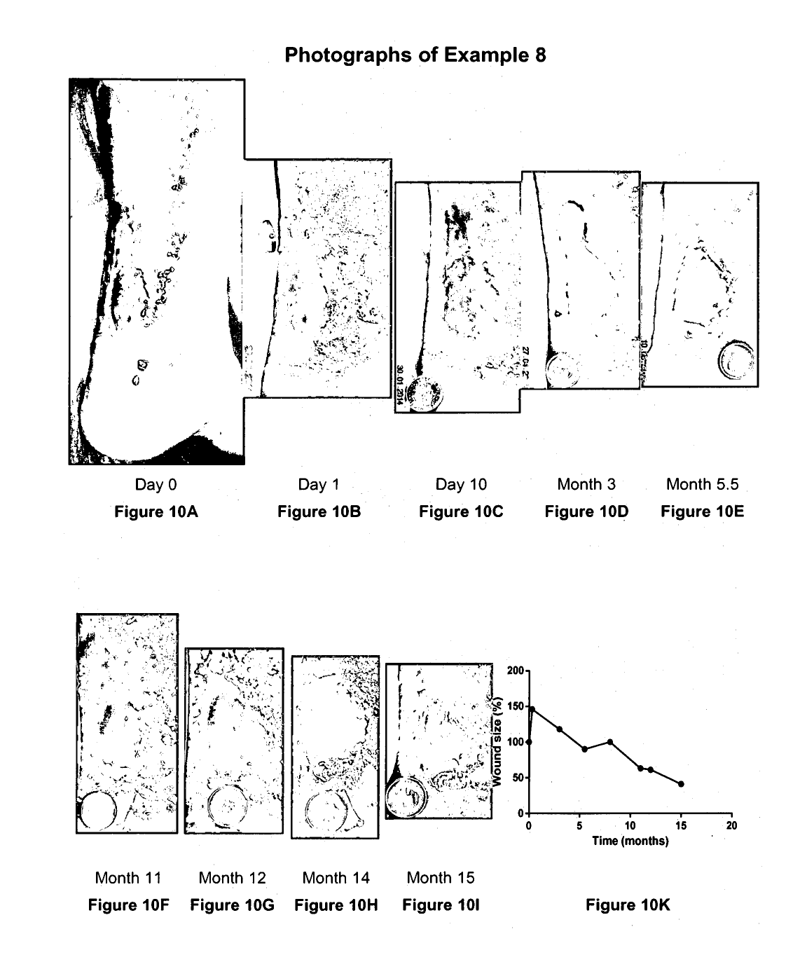

Photographs of the wound at Day 0 (prior treatment), Day 10, Month 3, Month 5.5, Month 11, Month 12, Month 14 and Month 15 of plasminogen treatment are shown in FIGS. 10A, 10B, 10C, 10D, 10E, 10F, 10G, 10H and 101, respectively. A graph reporting the wound size percentages during the plasminogen treatment (including its interruption between day 14 and Day 28) is shown at FIG. 10J.

Headings are included herein for reference and to aid in locating certain sections. These headings are not intended to limit the scope of the concepts described therein, and these concepts may have applicability in other sections throughout the entire specification Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1-47. (canceled)

48. A method for promoting the healing of a wound in a human subject, wherein said method comprises the local administration, to the subject, of at least one dose of plasminogen or a biologically active variant thereof, of about 2 mg to about 30 mg, wherein the local administration is intracutaneous, subcutaneous, intramuscular or topical administration.

49. The method of claim 48, wherein the local administration is subcutaneous or topical administration.

50. The method of claim 49, wherein the local administration is performed around the wound, in the vicinity of the wound or in the wound.

51. The method of claim 49, wherein the local administration consists of several injections performed within about 0.5 cm of the wound.

52. The method of claim 48, wherein the wound is an open wound.

53. The method of claim 48, wherein the wound is a chronic wound or an acute wound.

54. The method of claim 48, wherein the dose contains about 2 mg, about 4 mg, about 5 mg, about 10 mg, about 15 mg, about 20 mg, about 25 mg, or about 30 mg of plasminogen or a biologically active variant thereof.

55. The method of claim 48, wherein the local administration is performed at a frequency of at least once a week.

56. The method of claim 48, comprising providing a medicament wherein the medicament comprises a plurality of doses of plasminogen or a biologically active variant thereof, for performing multiple administrations until the wound size is reduced by at least 50%.

57. The method of claim 56, wherein the medicament comprises a plurality of doses of plasminogen or a biologically active variant thereof, for performing multiple administrations until the wound size is reduced by at least 90%.

58. The method of claim 48, wherein the plasminogen or biologically active variant thereof is in a solution at a concentration from about 1 mg/ml to about 20 mg/ml.

59. The method of claim 58, wherein the concentration is of about 1 mg/ml, about 5 mg/ml, about 10 mg/ml, about 15 mg/ml or about 20 mg/ml.

60. The method claim 48, wherein the wound has an area, and wherein the dose of plasminogen or a biologically active variant thereof is proportional to said area.

61. The method of claim 60, wherein the dose is expressed in mg and corresponds to about 2.5 times the wound area expressed in cm².

62. The method of claim 48, wherein the wound has an area, and wherein the dose is about 10 mg when said wound area is less than 4 cm²; the dose is about 20 mg when said wound area is from 4 cm² to 8 cm², or the dose is about 30 mg when said wound area is greater than 8 cm².

63. The method of claim 48, wherein the wound has a circumference, and wherein the dose of plasminogen or a biologically active variant thereof is proportional to said wound circumference.

64. The method of claim 63, wherein the dose is expressed in mg and corresponds to about 1.5 times the wound circumference expressed in cm.

65. The method of claim 51, wherein said several injections consist of 2 to 30 injections.

66. The method of claim 48, wherein the wound is a chronic wound selected from diabetic foot ulcers, diabetic wounds, pressure sores, pressure ulcers, tympanic membrane perforations, burn wounds, incision wounds, venous leg ulcers, arterial leg ulcers, vascular wounds and lesions.
